(19) United States
(12) Patent Application Publication
(10) Pub. No.: US 2009/0012473 A1
(43) Pub. Date: Jan. 8, 2009

(54) MEDICAL DEVICE FOR PLACEMENT ON THE SKIN OF A PATIENT

(76) Inventors: Marianne Stettler, Stallikon (CH); Marcel Hunn, Langenthal (CH); Christoph Walter, Derendingen (CH)

Correspondence Address: Dorsey & Whitney LLP, IP Department, ATTN: Disetronic Licensing AG, 50 South Sixth Street, Suite 1500, Minneapolis, MN, 55402-1498, US

(21) Appl. No.: 12/169,935

(22) Filed: Jul. 9, 2008

Related U.S. Application Data
(63) Continuation of application No. PCT/CH2006/000711, filed on Dec. 18, 2006.

(30) Foreign Application Priority Data
Jan. 10, 2006 (CH) 35/06

(51) Int. Cl. A61M 5/32; A61M 1/00

(52) U.S. Cl. 604/151; 604/180

(57) ABSTRACT

The application relates to portable medical devices that can be placed on a patient's skin and that include a silicone-based adhesive for attaching the device to the skin removeably or releasably. In preferred embodiments, the adhesive is a silicone pressure-sensitive adhesive (PSA) or a silicone soft-skin adhesive (SSA).

### CROSS-REFERENCE TO RELATED APPLICATIONS

This application is a continuation of International Patent Application No. PCT/CH2006/000711, filed on Dec. 18, 2006, which claims priority to Swiss Patent Application No. 35/06, filed on Jan. 10, 2006, the entire contents of both of which are incorporated herein by reference.

### BACKGROUND

The present invention relates to devices for delivering, administering, injecting, infusing or dispensing a substance, and to methods of making and using such devices. More particularly, it concerns portable medical devices which include a silicone-based adhesive for contacting and adhering to skin for removeably fixing or attaching the medical device on a human body.

Today's portable medical devices, for example infusion sets for subcutaneous administration of liquid medicines, typically have an acryl-based adhesive with which the devices are fixed to and/or on the skin of a patient. In continuous or long-term use, these adhesives can give rise to skin allergies and/or irritation in patients so that the therapy has to be interrupted. This is particularly troubling in serious cases in which continuous or generally continuous therapy is necessary.

### SUMMARY

An object of the present invention is to provide portable medical devices which can be worn by patients over a prolonged time without giving rise to skin allergies and/or skin irritation.

In one embodiment, a portable medical device according to the present invention includes a silicone-based adhesive for releaseably or removeably fixing the device on the skin.

In a preferred embodiment, the present invention comprises a medical device for the subcutaneous administration of a liquid medicine, e.g. an infusion set for the subcutaneous administration of insulin. Infusion sets for the subcutaneous administration of liquid medicines such as insulin which, in accordance with the invention can be releaseably fixed on the skin of a patient by a silicone-based adhesive are disclosed in, for example, EP 0 956 879 and WO 2004/026375. In some preferred embodiments, the medical device is a disposable infusion pump which, after emptying of the medicine reservoir contained therein, is removed from the surface of the skin by the patient and disposed of. The silicone-based adhesives are also suitable for fixing medical devices which include an implantable sensor for measuring a physiological parameter, e.g. blood sugar value or the interstitial glucose value.

Devices according to the present invention, e.g. infusion sets or infusion pumps, are distinguished in that, compared to the devices in the state of the art, they have increased skin compatibility (high biocompatibility) for patients and can thus be used in long-term therapies without causing allergies or irritation.

In a further preferred embodiment, a medical device in accordance with the present invention is an infusion pump, e.g. an insulin pump. Infusion pumps which in accordance with the invention can be releaseably or removeably fixed on the skin of a human being by a silicone-based adhesive are described in, for example, U.S. patent application 20040059316 and U.S. Pat. No. 6,740,059.

In some preferred embodiments, the silicone-based adhesive is part of a plaster which is disposed on an underside of the medical device. In some embodiments, the plaster may extend over the entire underside of the infusion set to afford good adhesion on the skin. The plaster may also cover only a part of the surface of the underside of the infusion set or be disposed in point form on the underside.

Silicone-based adhesives suitable and/or preferred for use in the present invention comprise silicone pressure-sensitive adhesives (PSA) and silicone soft skin adhesives (SSA).

Silicone adhesives are non-sensitising adhesives, that is to say adhesives which have good biocompatibility. There are two classes, silicone pressure-sensitive adhesives (PSA) and silicone soft skin adhesives (SSA). Silicone adhesives are distinguished in that adhesion is maintained when moisture is involved, e.g. perspiration, and also at different temperatures. In addition, the silicone adhesives have good moisture and air permeability as well as a long wearing life. By virtue of these properties, silicone adhesives are suitable for fixing medical devices on the skin of a patient.

### BRIEF DESCRIPTION OF THE DRAWINGS

FIG. 1 shows the structure of silicone pressure-sensitive adhesives;

FIG. 2 shows the structure of silicone soft skin adhesives (SSA); and

FIG. 3 shows an embodiment of a medical device, e.g. an infusion set, according to the present invention.

### DETAILED DESCRIPTION

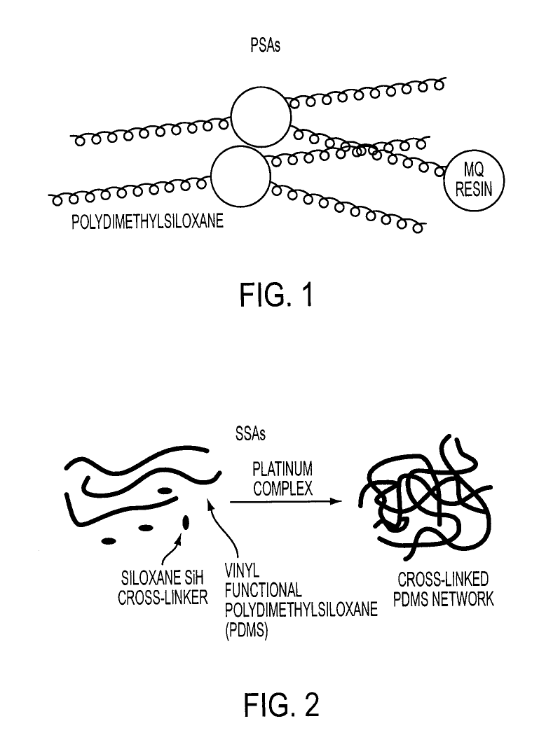

FIG. 1 shows the structure of silicone pressure-sensitive adhesives (PSA). Those adhesives are based on the resin-in-polymer principle which includes the condensation of dimethiconol to MQ resin. Dimethiconol is a low to medium viscous silanole polydimethylsiloxane (PDMS). MQ resin is a soluble three-dimensional silicate network of two different subunits which both contain silicon. Dimethiconol is the viscous component of the adhesive and is responsible for the visco-elastic behaviour and influences the moistenability and applicability of the adhesive. The resin acts as a stabilising element and is also responsible in terms of elasticity and enhancing the adhesive force. Silicone PSA's can be dissolved in heptane, ethyl acetate or volatile silicone liquids.

FIG. 2 shows the structure of silicone soft skin adhesives (SSA). SSA's consist of two parts and include no solvent. They are based on an interlaced elastomeric structure. Cross-linking is the result of a reaction between vinyl functional polydimethylsiloxanes (PDMS) and hydrogen functional siloxane. The process can be catalysed by a platinum complex and the result is a cross-linked polymer. They are rather gel-like by virtue of their different kind of structure. They have somewhat different properties from PSA insofar as they are less elastic, easily volatile and have greater moistening properties. Viscosity is very low, the dissolution properties are very pronounced.

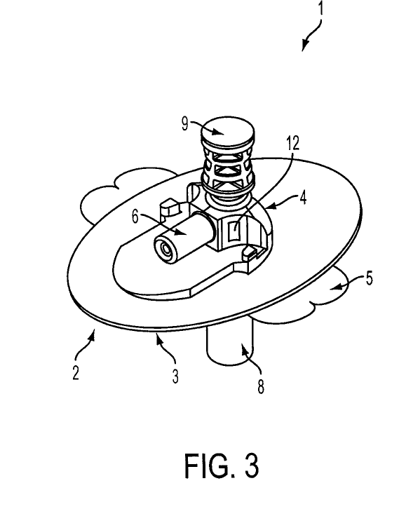

FIG. 3 shows an infusion set according to the present invention. The infusion set may be used for the administration of a liquid active substance into the subcutaneous tissue of a patient before application to the skin of a patient. On its underside 2 the infusion set 1 has a plaster 3 which includes the silicone-based adhesive for fixing or attaching the set to or on the skin of a patient. Prior to use the plaster 3 is covered by a film which includes a gripping portion 5 for more easily removing the film. It is possible to see on the top side of the infusion set 1 a hub 4, by which a connection is made with, for example, an external infusion pump. The connection of the hub 4 to the infusion pump can be implemented by a releasable coupling of the hub 4 to a tube connected to the pump. Disposed at the end of the tube which is connected to the hub 4 is a connecting portion which allows releasable coupling to the hub 4. In some preferred embodiments, the releasable coupling is a latching connection. The coupling of the tube is effected by the cylindrical extension 6 of the hub 4. The interior of the cylindrical extension forms a fluid passage through which the liquid to be delivered is passed into the body of a patient. The infusion set 1 includes on the underside a cannula which is introduced into the subcutaneous tissue of a patient. The cannula is at least partially in the form of a hollow needle to allow a flow of liquid. In some preferred embodiments, the cannula is a soft plastic cannula. Prior to application the cannula is protected by a needle protector 8. If the cannula involves a soft plastic cannula it may be introduced into the skin by a metal needle. The metal needle has a grip 9 which permits the patient to remove the metal needle after application of the set. In some embodiments, the infusion set or other medical device according to the present invention may comprise a sensor 12 for measuring a physiological parameter.

Application of the infusion set 1 to the skin comprises the following steps: the patient removes the film of the plaster 3 and the needle protector 8 and, with the metal needle having the grip 9, pierces the skin and places the infusion set on the skin. Thereafter, the patient pulls the metal needle out of the set by using the grip 9 and connects the infusion set to an infusion pump by a coupling of a tube. The set is now ready for the administration of liquid active substances.

1. A portable medical device for placement on the skin of a patient, the device comprising a silicone-based adhesive for releasably fixing the device on the skin.

2. The medical device according to claim 1, wherein the device is for the subcutaneous administration of a liquid medicine.

3. The medical device according to claim 2, wherein the device is a portable insulin pump.

4. The medical device according to claim 2, wherein the device is an infusion set for the subcutaneous administration of insulin.

5. The medical device according to claim 1, wherein the device comprises a sensor for measuring a physiological parameter.

6. The medical device according to claim 5, wherein the sensor is an electrochemical sensor for measuring a blood sugar value or an interstitial sugar content.

7. The medical device according to claim 1, wherein the adhesive is part of a plaster disposed on a surface of the medical device, said plaster for contacting the skin.

8. The medical device according to claim 7, wherein the surface is at least a portion of an underside of the device.

9. The medical device according to claim 8, wherein the surface is substantially flat.

10. The medical device according to claim 1, wherein the silicone-based adhesive is one of a silicone pressure-sensitive adhesive (PSA).

11. The medical device according to claim 1, wherein the silicone-based adhesive is a silicone soft skin adhesive (SSA).
